(19) United States
(12) Patent Application Publication
(10) Pub. No.: US 2013/0197320 A1
(43) Pub. Date: Aug. 1, 2013

(54) ULTRASONIC DIGITAL COMMUNICATION OF BIOLOGICAL PARAMETERS

(71) Applicants: David E. Albert, Oklahoma City, OK (US); James J. Lewis, Spokane, WA (US); Kim Norman Barnett, Mt. Tamborine (AU); Bruce Richard Satchwell, Carrara (AU)

(72) Inventors: David E. Albert, Oklahoma City, OK (US); James J. Lewis, Spokane, WA (US); Kim Norman Barnett, Mt. Tamborine (AU); Bruce Richard Satchwell, Carrara (AU)

(21) Appl. No.: 13/752,048

(22) Filed: Jan. 28, 2013

Related U.S. Application Data
(60) Provisional application No. 61/591,183, filed on Jan. 26, 2012.
(60) Provisional application No. 61/635,915, filed on Apr. 20, 2012.

(51) Int. Cl. A61B 5/00; A61B 5/145; A61B 5/021; A61B 5/01

(52) U.S. Cl. CPC A61B 5/0015; A61B 5/01; A61B 5/14532; A61B 5/021; A61B 5/14542
USPC 600/301; 600/300; 600/549; 600/365; 600/485; 600/364; 367/138

(57) ABSTRACT

Medical sensing devices and systems that transmit digital data from a first device via an ultrasonic digital modem to a receiver such as a smartphone. Methods of transmitting digital biological data by ultrasound are also described.

### CROSS REFERENCE TO RELATED APPLICATIONS

This patent application claims priority to U.S. Provisional Patent Application No. 61/591,183, filed Jan. 26, 2012, titled “ULTRASONIC SOFTWARE MODEM FOR MEDICAL DEVICES” and U.S. Provisional Patent Application No. 61/635,915, filed Apr. 20, 2012, titled “ULTRASONIC DIGITAL MODEM,” each of which is herein incorporated by reference in its entirety.

This material may be related to U.S. patent application Ser. No. 12/796,188, filed Jun. 8, 2010, titled “HEART MONITORING SYSTEM USABLE WITH A SMART PHONE OR COMPUTER,” now Publication No. US-2011-0301435-A1 and U.S. patent application Ser. No. 13/108,738, filed May 16, 2011, titled “WIRELESS, ULTRASONIC PERSONAL HEALTH MONITORING SYSTEM,” now Publication No. US-2011-0301439-A1, each of which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference

### FIELD

This patent application discloses inventive concept(s) related generally to systems, methods and devices, including hardware, firmware and software, for connecting medical devices having one or more sensors connected to a microprocessor and sound output to ultrasonically communicate with mobile communications and/or computing devices such as smartphones, tablets and computers.

### BACKGROUND

A large number of consumer products include the capability of providing sound outputs, including simple “beeps” and buzzers that may be used to communicate in the audible range to a user about the status of the device. Such devices typically include a tone generator (e.g., a piezoelectric speaker) and a controller (microcontroller) that may control output from the tone generator. Although it is possible to add additional elements, including circuitry, antenna, and signal processing elements to these devices to enable them to wirelessly communicate (typically via electromagnetic means) with other electronic devices, such modifications may add substantial cost and complexity. It would be of substantial benefit provide devices, methods and systems (including specifically firmware, software, and/or hardware) that can ultrasonically transmit information, and particularly digital information, using ultrasound rather than electromagnetic signals to another device, and particularly a telecommunications device that may store, process, analyze, and/or retransmit the information.

Consumer medical devices (e.g., medical devices for personal use, such as thermometers, glucose monitors, blood pressure cuffs, pulse oximeters) are one example of a technology that would benefit from a simple, reliable and cost effective way to transmit data ultrasonically to a telecommunications device. For example, many medical devices include a digital display to present output. This digital information is not usually transmitted beyond the device. However, in many instances it may be beneficial to transmit the digital medical health information to one or more locations so that the medical information may be accessed and/or manipulated by others. For example, it may be useful for a patient to record and provide access to detected health information (e.g., blood pressure, blood sugar, temperature, telemetry, etc.) to medical professionals. Access may be provided by uploading the medical information to a server and/or website; the website may be used to store, provide remote access to the user and/or qualified medical professionals, or analyze the health information.

Currently available or proposed systems capable of transmitting health information from a medical device typically require a dedicated wireless transmitter or act though a dedicated sub-system for transferring and/or uploading such health information. In addition to requiring additional devices and systems, this has also proven expensive both in materials and power requirements.

Described herein are methods, devices, and systems for using (or adapting for use) one or more widely available computing devices including a microphone (e.g., a telecommunications device), such as smart phones, tablet computers, portable computers or desktop computers, to receive and send digital health information that has been encoded by an application device into an ultrasonic signal that can be heard by the telecommunications device and then stored, transmitted and/or analyzed by the telecommunications device.

U.S. patent application Ser. No. 12/796,188, filed Jun. 8, 2010, titled “HEART MONITORING SYSTEM USABLE WITH A SMART PHONE OR COMPUTER,” now Publication No. US-2011-0301435-A1 and U.S. patent application Ser. No. 13/108,738, filed May 16, 2011, titled “WIRELESS, ULTRASONIC PERSONAL HEALTH MONITORING SYSTEM,” now Publication No. US-2011-0301439-A1, describe ECG monitors that convert ECG data into ultrasound signals that can be received by a telecommunications device such as a smartphone and then stored, analyzed, and/or displayed. The instant application extends these teaching to include digital medical devices such as thermometers, blood pressure sensors, blood sugar monitors, pulse oximeters and the like, in which the biological parameters can be interpreted and digitally represented before transmitting. In addition, described herein are methods and systems for adapting or retrofitting any existing microprocessor that control a sound-generating source (e.g., buzzer), so that it can be used to reliably transmit digital ultrasonic information.

### SUMMARY OF THE DISCLOSURE

In general, described herein are devices, systems and methods for ultrasonically transmitting digital data from a device having a microprocessor and a transducer capable of delivering ultrasonic frequencies (i.e., piezo speaker). The digitally transmitted data may be received by a receiving device having a microphone, such as a telecommunications device (e.g., a personal telecommunications device, phone such as an iphone, DROID, or other smartphone, iPad or other personal computers, PDAs, or the like). The digital information transmitted may be encoded and/or encrypted as described in greater detail below.

In particular, described herein are methods for controlling the transmission of ultrasonic digital information (e.g., medical/biological parameters or information) so that it can be reliably transmitted either with or without confirming receipt of the information (e.g., duplex or half-duplex communication.). In some variations the transmitted ultrasound information is encoded in two ultrasound frequencies (e.g., a frequency corresponding to digital zero, and a frequency corresponding to digital one). In some variations a third (or additional) frequency is used to transmit a calibration tone that can be used by the receiver. A calibration tone may be at an frequency separate from the frequencies representing digital one/zero, and may be constantly emitted, emitted between data transmission, or emitted concurrent with data transmission. In some variations the calibration tone is constant; in some variations a portion of the calibration signal/tone is constant (e.g., amplitude), but the tone is configured to indicate timing (e.g., counting down to the next data transmission). A receiving device (e.g., telecommunications device) may use the calibration tone/signal to calibrate the receipt of information at the digital frequencies (e.g., digital zero/digital one).

As mentioned, it may be useful to provide ultrasonic communication between the receiving device (e.g., a telecommunications device such as a smartphone or computer) and ultrasonic transmission devices. For instance, it would be helpful to implement a half-duplex protocol so that the telecommunications device (e.g., smartphone/computer) could provide acknowledgement (ACK) to the sensing device (source device or ultrasonic transmission device) that the data has been successfully received (with correct CRC) and to stop re-transmitting that data. Another use of this half-duplex protocol would be to configure the ultrasonic transmission device by sending parameters or information such as calibration data, personal information, etc. from the receiving device (e.g., telecommunications device). As mentioned above, the ultrasonic transmission device may transmit a calibration signal at a third (or more) frequency that is separate from the digital ultrasonic frequencies, which may be received and used by the receiving (e.g., telecommunications) device.

In some variations the microcontroller of the ultrasonic transmission device is configured for duplex (e.g., half-duplex) configuration by receiving an acknowledgement signal from the same transducer (e.g., piezo) that is used to transmit ultrasonically. For example, after transmitting from the transducer for a predetermined period of time, the microcontroller may be configured to “listen” to the transducer to determine if is receiving an acknowledgement signal. Although a transducer for transmission of an ultrasonic signal may not be specifically adapted for receipt of an ultrasonic signal, the inventors have empirically observed receipt of ultrasound signals by a emitting transducer. The acknowledgement signal may be a single pulse, a train of pulses, or a pattern of pulses.

Any of the variations described herein may be configured to operate as a simplex system (e.g., transmission only). When operating as a simplex system, the ultrasonic transmission device may be configured to repeatedly transmit the information for a predetermined amount of time and/or number of repeats. In some variations, the ultrasonic transmission device is configured to continuously transmit digital ultrasonic information for seconds, minutes, or hours.

Also described herein are ultrasonic digital transmitters configured as ultrasonic modems having digital modem protocols and logic for transmitting digital information ultrasonically to a receiver, which may be configured as a telecommunications device. Thus, the systems may be configured with ultrasonic modem protocols (logic) for structuring the digital data signal, including a header portion and/or data portion. The signal may be broken into packets or any other measure of digital information (byte, packet, words, etc.). The signal may be configured to include error correction code(s).

For example, described herein are microcontroller configured as ultrasonic modems. In some variations the microcontrollers include logic (e.g., hardware, software, firmware, or some combination thereof) that permits the device to drive ultrasonic transmission of data from a speaker (e.g., piezoelectric speaker element). Methods of configuring or adapting a microcontroller to operate as an ultrasonic modem are also described. For example, in some variations a microcontroller may be programmed to operate as an ultrasonic modem.

Also described herein are receivers configured to receive ultrasonic digital data acoustically transmitted by an ultrasonic digital modem. In general, a telecommunications device (e.g., smartphone) may be configured to act as a receiver to receive ultrasonic digital data. Thus, a telecommunications device may include hardware, software, and/or firmware configured to receive, decode, interpret, display, analyze, store and/or transmit data sent by ultrasonic transmission from a digital ultrasonic modem. In some variations logic (e.g., client software and/or firmware, applications, etc.) may be executed on the telecommunications device so that it may act as a receiver for the digital ultrasound data. Thus, described herein is executable logic for receiving and interpreting (e.g., decoding) data transmitted by digital ultrasonic modem, and devices including executable logic for receiving and interpreting (e.g., decoding) data transmitted by digital ultrasonic modem executable logic. In general this executable logic is configured to be stored in a non-transient medium so that it may be executed later (or repeatedly).

Further described herein are specific devices and system configured to include digital ultrasonic modems. Any of these devices may include a source of the digital information (e.g., device such as a medical device (e.g., thermometer, pulse oximiter, etc.), a sound transducer (e.g., a speaker capable of emitting ultrasound signals) and a controller (e.g., microcontroller) configured to encode digital information from the source of digital information as an ultrasound signal to be transmitted by the sound transducer. In some variations the sound transduce is configured to emit both audible (e.g., lower than ultrasound) sounds (to buzz, beep and the like within normal human hearing range) as well as emitting in the ultrasound frequency (e.g., greater than 17 KHz).

In one example described herein a Texas Instrument's AFE4110 digital thermometer has been modified/retrofitted as described herein to ultrasonically digitally encode and transmit the temperature data ultrasonically (as an ultrasonic pressure wave through the air) to a telecommunications device (e.g., a smartphone) located some distance from the thermometer. The microcontroller of the device (an MSP430 type controller from Texas Instruments) has been configured as an ultrasonic modem for transmission of ultrasonic digital data executing firmware/software causing the microcontroller to encode (via the microprocessor) a temperature data signal for transmission on a connected piezoelectric speaker. The speaker may be the same speaker that is preset in the thermometer and used for audibly (e.g., with the normal audible range for humans) notifying the user that the temperature is stable. Thus, the thermometer may be retrofitted to include the digital ultrasound modem at very low cost by executing control logic in the microcontroller to process data from the thermometer and transmit the encoded signal on the piezoelectric speaker in the ultrasonic frequency range (e.g., >17 KHz).

For example, in some variations, described herein are medical sensing devices and systems including such devices that use ultrasound to digitally transmit biological parameters received by the medical sensing device to one or more telecommunications devices (e.g., a smartphone) where the information can be further processed and/or transmitted on. The executable logic may also be referred to as an adapter for adapting medical sensing devices so that they may ultrasonically transmit biological parameter information to a telecommunications device for further processing. Also described are systems and/or subsystems for use with a telecommunications device so that the telecommunications device can receive and translate an ultrasonically encoded health metric information signal. These subsystems may include client software (e.g., applications) to be run on the telecommunications device (e.g., phone) to translate the ultrasonic health information (or biological parameter) signal into a digital signal that can be uploaded, stored, and/or analyzed by the telecommunications device.

A medical sensing device may be any device for receiving biological parameters, such as patient vitals. The biological parameters may also be referred to as biometric data. For example, a medical sensing device may be a thermometer, blood pressure transducer, glucose monitor, pulse oximeter, etc. The Medical sensing devices or systems referred to herein are typically digital systems because they may display a numeric (e.g., digital) representation of the biological parameter. For example, the devices may convert analog biological parameters (e.g., temperature, blood sugar, blood pressure or any other health metric information) into digital signals that may be displayed or otherwise presented to the user. For example, a medical sensing system may include a digital thermometer for taking a subject's temperature, a blood cuff for presenting patient blood pressure, a blood sugar (glucose) monitors, a pulse oximeter, or the like, including combinations of these devices. Medical sensing systems or devices for home use are of particular interest, and especially those having sensors that monitor or collect biological parameters from patients and present the information on a display.

As used herein biological parameters or information may include any patient information that is processed, sensed, and/or calculated by a medical sensing system, and particularly digitally encoded biological parameters. For example, biological parameters may include temperature, blood pressure, blood sugar level, pH, oxygenation, pulse rate, respiratory rate, or any other biological measurement, particularly those relevant to medical case, including diagnosis and health monitoring.

As used herein telecommunications devices includes smartphones (e.g., iPhone™, droid™ or other personal communications devices), tablet computers (e.g., ipad™, tablet PCs, or the like), and/or desktop computers that include (or may be adapted to include) a microphone capable of receiving ultrasonic sound. A telecommunications device may include logic for translating the digital signal encoded by the ultrasonic sound into a digital signal that can be displayed, uploaded/transmitted, stored, and/or analyzed.

Thus, in some variations, described herein are medical sensing devices for ultrasonically transmitting digital biological parameters. In some variations the device may include: a sensor for detecting a biological parameter from a patient; a processor for encoding a digital representation of the biological parameter as an ultrasound sound signal; and an ultrasonic transducer for transmitting an ultrasonic sound signal from the processor.

For example, a medical sensing device may include a transducer for transducing a biological parameter (e.g., temperature sensor, pressure sensor, etc.). The device may also include a controller (e.g., microcontroller) for processing signals from the sensor(s). The processor may include a signal generator that generates a signal from sensed and/or processed patient biological parameter information; the signal may be encoded for transmission. The signal may be encoded as a digital packet (e.g., words, bytes, etc.). For example, the signal may include a start bit, stop bit, information bit(s) identifying the type or source of the biological parameter (e.g., packet identifier), a digital representation of the biological parameter and in some variations a cyclic redundancy check (CRC) portion. In some variations, the signal (including the biometric measurement or data portion) can have a time and/or date stamp.

Thus, in some variations, the system or devices may be configured so that the measurement is made at time x and stored on the device (e.g., thermometer, glucometer, etc.) and transmitted to the telecommunications device (e.g., smartphone or tablet) ultrasonically at a later time, and eventually uploaded (e.g., to the cloud). In some variations, several time/date stamped measurements may be stored on a device and could be transmitted together in a burst to the telecommunications device. As described in greater detail below, although the device may be primarily one-way (e.g., sending data from the biometric device to the telecommunications device) in some variations the devices may be configured to receive at least a confirmation signal and/or an indicator of the proximity of the telecommunications device. In some variations the ultrasonic transducer may also be configured to receive a confirmation signal (ACK) from the telecommunications device. Confirmation may indicate that the telecommunications device received a sent message (data) or that the telecommunications device is ready to receive the sent data, or both.

The ultrasonic transducer may be any appropriate transducer, including a piezo crystal transducer.

In some variations, a system for ultrasonically transmitting digital biological parameter includes: a medical sensing device having: a sensor for detecting a biological parameter, a processor for encoding a digital representation of the biological parameter as an ultrasound sound signal, and an ultrasonic transducer for transmitting the ultrasonic sound signal; and client control logic configured to be executed by a telecommunications device and to receive the ultrasonic sound signal and convert it back to a digital representation of the biological parameter.

The processor may convert the digital biological parameter signal (which is typically a numeric value) into an ultrasonic signal by the use of any appropriate signal processing technique, including, but not limited to, frequency-shift keying.

The client control logic may also be referred to as software (though it may be software, hardware, firmware, or the like), or a client application. The client control logic may execute on a telecommunications device. The client control logic may also include components for passing the digital representation of the biological parameter on to other devices, e.g., uploading it to a website or server, for example. In some variations the client control logic may be configured to display or otherwise present the information locally on the telecommunications device.

Also described herein are systems for transmitting a digital health parameter, the system comprising: an ultrasonic transducer, wherein the ultrasonic transducer is capable of transmitting signals in an open-air environment at frequencies above about 17 KHz (e.g., 19 KHz, or centered around 20 KHz); and a signal generator configured to generate an ultrasonic signal corresponding to a digital representation of a biological parameter, wherein the identifier is associated with at least one frequency above about 17 KHz (e.g., 19 KHz, or centered around 20 KHz).

As an example, described herein are digital thermometer to ultrasonically transmit digital temperature information to a telecommunications device for further processing and transmission. The digital thermometer may include: a temperature sensor for sensing patient temperature; a signal generator for generating a signal corresponding to a digital representation of the patient temperature; and an ultrasonic transducer for transmitting the digital representation of the patient's temperature as an ultrasonic signal comprising one or more frequencies above 19 KHz.

Method of operation, including methods of sending digital ultrasonic biological parameter information and methods of receiving this information by a telecommunications device are also described. For example, described herein is a method of wirelessly receiving digital biological parameters from a medical sensing device on a telecommunications device, the method including the steps of: receiving on a telecommunications device an ultrasonic signal encoding a digital representation of a biological parameter from a medical sensing device; and converting the ultrasonic signal into an electronic signal. In some variations, the method includes the step of transmitting the electronic signal to an external site. In some variations the method includes the step of determining from the electronic signal the type of biological parameter. As mentioned, the ultrasonic signal may be encoded to identify the type of the biological parameter signal. For example, the signal may be encoded to indicate that it is a heart rate, blood pressure measure, temperature, etc.

Also described herein are methods of wirelessly transmitting digital biological parameters from a medical sensing device to a telecommunications device, the method comprising: sensing a biological parameter; creating a digital representation of the biological parameter; and transmitting the digital representation of the biological parameter as an ultrasonic signal.

Further described herein are medical sensing devices for detecting a biological parameter, determining a digital representation of the biological parameter, and ultrasonically transmitting the digital representation of the biological parameters as an inaudible sound transmission. Such devices may include: a sensor for detecting a biological parameter from a subject; a processor configured to receive the biological parameter, determine a representative value from the biological parameter, and digitally encode the representative value as a digital ultrasound signal, wherein the digital ultrasound signal is encoded using a first frequency corresponding to digital zero and a second frequency corresponding to digital 1, wherein the first and second frequencies are each greater than 17 kHz, further wherein the digital ultrasound signal includes a header portion, and a data portion; and an ultrasonic transducer comprising an ultrasound emitter for transmitting the digital ultrasound signal, wherein the processor is configured to drive the ultrasonic transducer to emit the digital ultrasound signal from the ultrasound emitter.

Any appropriate sensor may be used, and particularly sensors configured to sense a biological parameter, such as: temperature, glucose, pulse oxygenation, or blood pressure.

In general, the processor is a microprocessor. As mentioned, the microprocessor may be adapted as an ultrasonic modem to encode biological information as ultrasonic digital data for transmission. For example, the processor may be configured to encode the biological data as digital information using a first frequency of approximately 18.5 kHz and the second frequency of approximately 19.5 kHz. The processor may be configured to digitally encode the digital ultrasound signal at any appropriate rate. For example, at approximately 10 cycles per bit, and/or to digitally encode the digital ultrasound signal at 200 bytes/second.

As mentioned above, in any of these variations, the processor may be further configured to send a calibration tone at a frequency. In some variations this calibration tone is a continuous tone, and the calibration tone is typically separate from the first and second frequencies (the “zero” and “one” frequencies) to indicate the presence of the device and signal strength.

The digital ultrasound signal may generally include an error correction code.

In general, the ultrasound emitter comprises a speaker; for example, the ultrasound emitter comprises a piezoelectric element.

Also described herein are systems for detecting a biological parameter, determining a digital representation of the biological parameter, and ultrasonically transmitting the digital representation of the biological parameters as an inaudible sound transmission, the system comprising: a medical sensing device having: a sensor for detecting a biological parameter, a processor configured to receive the biological parameter, determine a representative value from the biological parameter, and digitally encode the representative value as a digital ultrasound signal using a first frequency corresponding to digital zero and a second frequency corresponding to digital 1, wherein the first and second frequencies are each greater than 17 kHz, and an ultrasonic transducer for transmitting a digital ultrasound signal; and client control logic configured to be executed by a telecommunications device and to cause the telecommunications device to receive the digital ultrasound signal and extract the representative value of the biological parameter from the digital ultrasound signal.

As mentioned above, the sensor may be configured to detect one or more of: temperature, glucose, pulse oxygenation, or blood pressure.

In general, the processor may be further configured to send a calibration tone at a frequency that is separate from the first and second frequencies; the calibration tone may be continuous or discrete and may indicate the presence of the device and signal strength. In some variations the calibration tone indicates the time to the next data transmission.

In general, the digital ultrasound signal may include a header portion, a data portion and an error correction code portion. The client control logic may comprise non-transitory computer-readable storage medium storing a set of instruction capable of being executed by a smartphone.

For example, described herein are digital thermometers to ultrasonically transmit digital temperature information to a telecommunications device for further processing and transmission, the digital thermometer comprising: a temperature sensor for sensing subject's temperature; a processor in communication with the temperature sensor and configured to generate a digital ultrasound signal of the subject's temperature, wherein the digital ultrasound signal is encoded using a first frequency corresponding to digital zero and a second frequency corresponding to digital 1, wherein the first and second frequencies are each greater than 17 kHz; and an ultrasonic transducer comprising an ultrasound emitter, wherein the processor is configured to drive the ultrasonic transducer to emit the digital ultrasound signal from the ultrasound emitter.

As with any of the device and systems described herein the first (zero) and second (one) frequencies may be any appropriate frequencies, including in particular frequencies in the inaudible (e.g., ultrasound) range. For example, the first frequency may be approximately 18.5 kHz and the second frequency approximately 19.5 kHz.

In some variations the processor is configured to send a calibration tone at a frequency that is separate from the first and second frequencies to indicate the presence of the device and signal strength.

Also described herein are methods of locally transmitting a representative value of a biological parameter using ultrasound, the method comprising: sensing a biological parameter from a subject; determining a representative value from the biological parameter; digitally encoding the representative value as a digital ultrasound signal, wherein the digital ultrasound signal is encoded using a first frequency corresponding to digital zero and a second frequency corresponding to digital 1, wherein the first and second frequencies are inaudible ultrasound frequencies; and driving an ultrasonic transducer near the patient to emit the digital ultrasound signal as an inaudible sound signal.

In general, sensing a biological parameter may comprise sensing any biological parameter or parameters, including one or more of: temperature, glucose, pulse oxygenation, or blood pressure.

Determining a representative value may comprises determining one or more of an average, a mean, a median, a maximum, a minimum, or a rate of change of the biological parameters. In some variations the biological parameter is on a relative scale (e.g., percent change) whine in some variations the biological parameter is on an absolute scale (e.g., temperature, pressure, concentration, etc.).

Digitally encoding the representative value may comprise encoding the digital ultrasound signal to include a header portion and a data portion (and an error correction code, which may be referred to as a CRC “portion” even though it may not be a discrete section). Digitally encoding the representative value may comprise digitally encoding the digital ultrasound signal at 10 cycles per bit; digitally encoding the representative value may comprise digitally encoding the digital ultrasound signal at 200 bytes/second.

Any of the methods described herein may include emitting a calibration tone at a frequency that is separate from the first and second frequencies. The calibration tone may indicate the presence of the device and signal strength. The calibration tone may be continuous.

Any of the variations described herein may include the step of confirming or acknowledging receipt of transmission. For example half-duplex communication including receipt of an acknowledgement (ACK) from the telecommunications device to the transmitting device. In some variations the method includes repeatedly driving the ultrasonic transducer to emit the digital ultrasound signal until a receipt confirmation is received. Alternatively, in some variations, the method includes repeatedly driving the ultrasonic transducer to emit the digital ultrasound signal for a predetermined period of time or number of repeats.

Also described herein are integrated microprocessors configured as an local ultrasonic data transmission device, the microprocessor comprising a non-transitory computer-readable storage medium storing a set of instruction for: receiving a value, digitally encoding the value as a digital ultrasound signal, wherein the digital ultrasound signal is encoded using a first frequency corresponding to digital zero and a second frequency corresponding to digital 1, wherein the first and second frequencies are inaudible ultrasound frequencies, adding a header portion to the digital ultrasound signal; and an ultrasonic transducer comprising an ultrasound emitter for transmitting the digital ultrasound signal.

### BRIEF DESCRIPTION OF THE DRAWINGS

FIG. 1 is a pictorial representation of the human range and thresholds of hearing from http://en.labs.wikimedia.org/wiki/Acoustics.

FIG. 2 is a pictorial representation of hearing loss with age from www.neuroreille.com/promenade/english/audiometry/audiometry.htm.

FIG. 3 is an audiogram illustrating the intensity and frequency of common sounds from www.hearinglossky.org/hlasurvival1.html.

FIG. 4A is a schematic representation of a system that is configured to ultrasonically transmit digital data encoding one or more biological parameter to a telecommunications device such as a smartphone.

FIG. 4B is a schematic representation of a system including a medical sensing device that is configured to ultrasonically transmit digital data encoding one or more biological parameter to a telecommunications device such as a smartphone.

FIG. 5 shows one variation of a digital signal that has been encoded using frequency key-shifting in an ultrasound range, as described.

FIG. 6 is an exemplary flowchart illustrating one method of transmitting encoded data as an ultrasound signal.

FIGS. 7A-7E are exemplary flowcharts of a method for transmitting a signal (e.g., packet transmission) as an ultrasound signal.

FIG. 8 shows one example of flowchart of a demodulator and packet decoder for a receiver configured to receive and decode data that is transmitted ultrasonically as discussed herein.

### DETAILED DESCRIPTION

In general, described herein are systems for ultrasonically transmitting digital information (e.g., digital representations of biological parameter information) from a first device to a telecommunications device that can then process and/or transmit the biological parameter information on.

For example, a system capable of ultrasonically transmitting digital biological parameter information may include a sensor for sensing a biological parameter (e.g., vital sign), a processor for configuring a digital representation of the biological parameter as a “digital” ultrasonic signal, and a transducer for transducing the ultrasonic signal so that it can be open-air transmitted to a telecommunications-capable device. The processor may part of, controlled by or in communication with a controller (e.g., a microcontroller). The telecommunications-capable device (telecommunications device) typically includes a receiver (audio receiver) able to receive an audio signal in the ultrasonic range, and a processor for converting the ultrasonic signal back into an electronic signal for further processing or transmission.

It is to be understood that the invention is not limited in its application to the details of construction, experiments, exemplary data, and/or the arrangement of the components set forth in the following description. The invention is capable of other embodiments or of being practiced or carried out in various ways. Also, it is to be understood that the terminology employed herein is for purpose of description and should not be regarded as limiting.

In the following detailed description of embodiments of the disclosure, numerous specific details are set forth in order to provide a more thorough understanding of the disclosure. However, it will be apparent to one of ordinary skill in the art that the concepts within the disclosure can be practiced without these specific details. In other instances, well-known features have not been described in detail to avoid unnecessarily complicating the description.

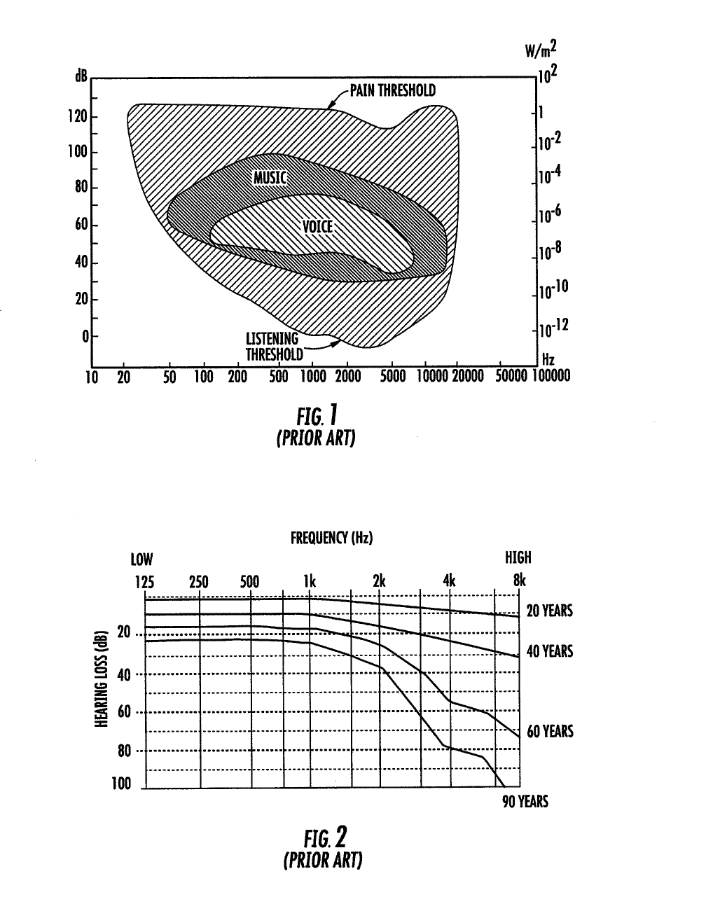

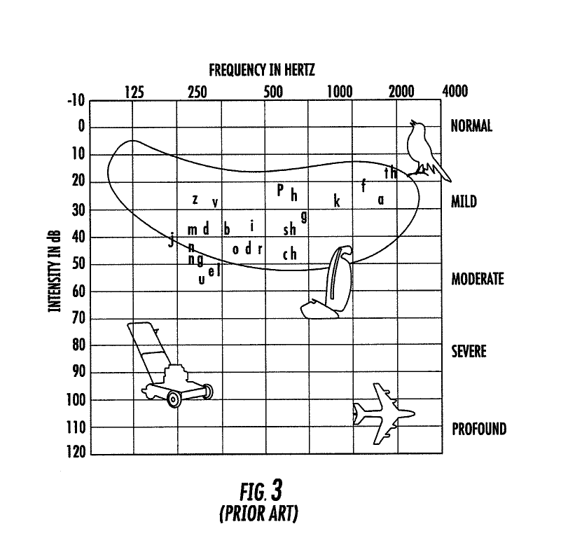

The human hearing range is often referred to as 20 Hz to 20 kHz. A maximum aural range in children, under ideal laboratory conditions, is actually as low as 12 Hz and as high as 20 kHz. However, as shown in FIG. 1, the threshold frequency, i.e. the minimum intensity detectable, rises rapidly to the pain threshold between 10 kHz to 20 kHz. Thus, sounds above about 16 kHz must be fairly intense to be heard. Almost immediately from birth, the threshold sound level for these higher frequencies increases. As shown in FIG. 2, an average 20 year old has lost about 10 dB in the 8 kHz range, while at age 90, the average person has lost over 100 dB at this frequency.

An example product using very high frequency sound is the Mosquito alarm, a controversial device emitting an intentionally annoying 17.4 kHz alarm and used to discourage younger people from loitering. Due to adult hearing loss at this frequency, it is typically heard only by people less than 25 years of age. Similarly, students make use of the adult hearing loss by using “mosquito” ringtones in the 15-17 kHz on their cell phones during school. The students can hear the “mosquito” ringtones while their adult teachers cannot. The term “ultrasonic” typically means above the range perceived by humans. However, as demonstrated, the upper limit of hearing frequency varies with individuals and with age generally. Because of the differences in this upper limit, the term “ultrasonic” is defined herein and in the appending claims to refer to “sound frequencies of 17 kHz or greater.”

Interestingly, however, there is very little ambient sound or noise above about 10 kHz. Referring to FIG. 3, most everyday sounds occur at frequencies below about 4 kHz. Thus, use of signals in the ultrasonic range is not only silent to those around, but also provides a very desirable signal to noise ratio (SNR).

Acoustic engineers safely assume that any frequency above about 20 kHz will have no effect on the perceived sound and they filter everything above this range. Sounds below 20 kHz but still in the ultrasonic range are of little concern, and standard sampling procedures have been established accordingly. It is generally understood that sampling an analog signal, whether a radio signal or audible sound signal, requires a sampling frequency fₛ such that fₛ/2 >f, wherein f is the sinusoid frequency. For this reason, sound systems are designed to sample the sound at the now standard sample rate of 44.1 kHz, set somewhat higher than the calculated Nyquist-Shannon sampling rate of 40 kHz for a 20 kHz sound upper limit. Actual demodulation of an FM narrow band signal in the ultrasonic range, using existing demodulation procedures, computers, telephones, cell phones, stereo sound systems, etc., would result in very poor reproduction of the original signal. This is unfortunate because, as discussed above, a carrier signal in the ultrasonic range would also have a very low signal to noise ratio due to the fact that there is very little natural “noise” at these higher frequencies.

The devices, methods and systems for measuring physiological signals (e.g., biological parameters) and transmitting digital information about those measurements wirelessly and soundlessly use ultrasonic signals having a much improved signal to noise ratio compared to traditional transtelephonic methods. Also provided are methods and algorithms to receive and demodulate the ultrasonic signals with excellent accuracy using existing computer and smart phone technology.

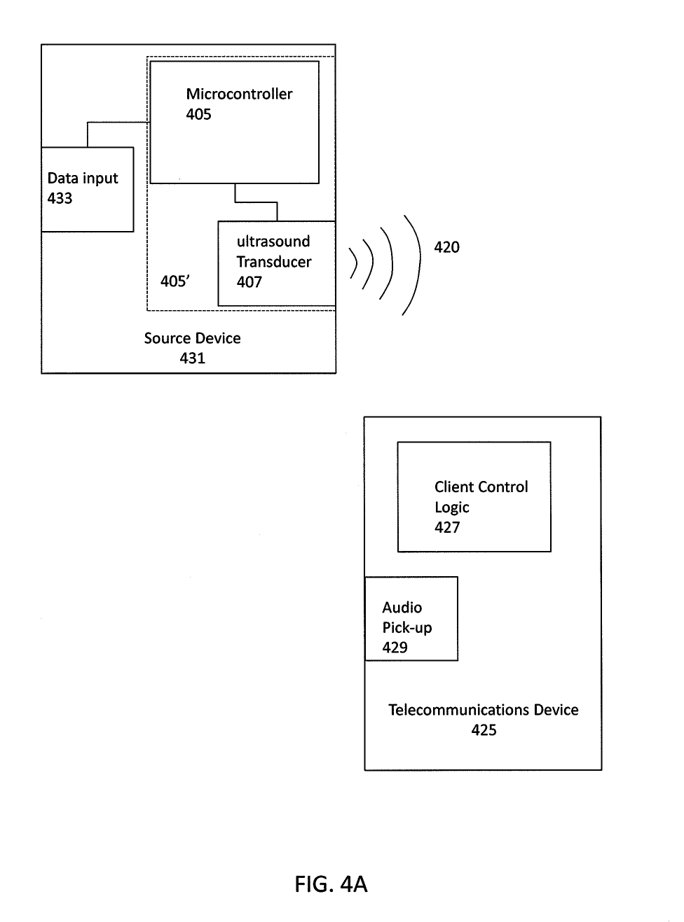

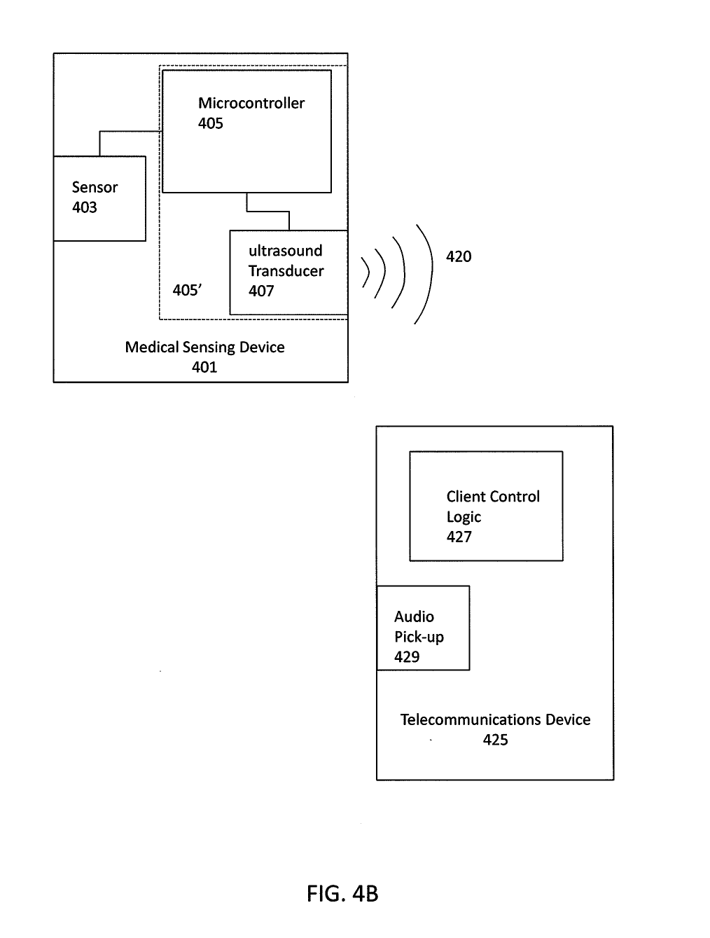

FIG. 4A shows a schematic overview of a system including a data input 433 (e.g., providing any sort of digital information) and a microcontroller 405. The microcontroller may include or be coupled with a processor for encoding a digital representation of a biological parameter, and this encoded signal may be converted to an ultrasound signal as described in more detail below. For example, the encoded signal may be transmitted ultrasonically by an ultrasonic transducer 407. In some variations the microprocessor and the transducer may be coupled together or formed as part of the same component 405′, alternatively, the microprocessor may include a piezo/speaker element. This ultrasonic signal 420 may then be received by a telecommunications device 425, including an audio pick up (receiver) 429. The telecommunications device 425 may run client control logic 427 preparing the telecommunications device to receive and translate the ultrasonic signal so that it can be processed, e.g., converting it back to an electronic signal, and interpreting which type of signal it is (e.g., pulse rate, temperature, etc.).

FIG. 4B shows a schematic overview of a system including a medical sensing device 401 (e.g., a thermometer, blood glucose monitor, or the like) that has a sensor 403 for detecting a biological parameter from a patient (e.g., temp, pulse rate, blood glucose, etc.) and a microcontroller 405. The microcontroller may include or be coupled with a processor for encoding a digital representation of a biological parameter, and this encoded signal may be converted to an ultrasound signal as described in more detail below. For example, the encoded signal may be transmitted ultrasonically by an ultrasonic transducer 407. This ultrasonic signal 420 may then be received by a telecommunications device 425, including an audio pick up (receiver) 429. The telecommunications device 425 may run client control logic 427 preparing the telecommunications device to receive and translate the ultrasonic signal so that it can be processed, e.g., converting it back to an electronic signal, and interpreting which type of signal it is (e.g., pulse rate, temperature, etc.).

Thus, medical sensing device 401 includes a sensor (or sensor assembly) configured to sense one or more physiological signals, such as temperature, pulse, pressure (e.g., blood pressure) or the like. The sensor may produce electrical signals representing the sensed physiological signals and these signals may be converted to a digital signal or signals that input to microcontroller or other associated components. This digital signal may typically be displayed on the device (not shown) and may also be electrically encoded as part of a digital signal that can then be ultrasonically encoded (e.g., by a technique such as frequency shift keying) to an ultrasonic sound and emitted from the device. The encoding of the signal may be performed by any appropriate circuitry, including, for example a microcontroller such as the MSP430 (e.g., the AFE4110 from Texas Instruments).

The center frequency may be selected from any appropriate ultrasonic frequency, including (but not limited to) 20 KHz. Typically the medical sensing devices described herein are configured as transmit only, so that data is transmitted to (but not received from) a telecommunications devices. In some variations, the medical sensing devices are configured to both send and receive ultrasonic (sound) frequency information. Further, in some variations, multiple channels (frequency channels) may be used.

In one embodiment, the ultrasonic signal has a center frequency in the range of from about 18 kHz to about 24 kHz. In another embodiment, the frequency modulated ultrasonic signal has a center frequency in the range of from about 20 kHz to about 24 kHz.

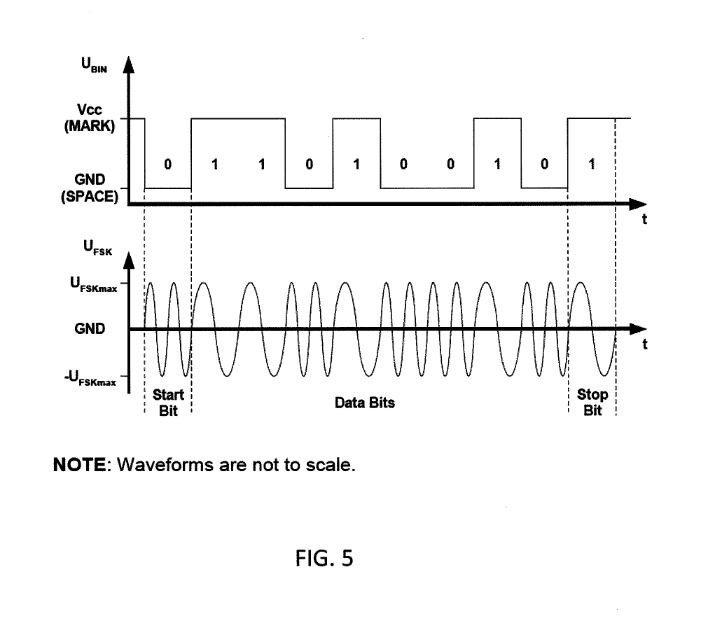

FIG. 5 shows one variation of a digital signal that has been encoded using key-shifting. In this variation the ultrasound signal is modulated at two different frequencies, one indicating high (“1”) and one indicating low (“0”). For example, the frequencies for 0 and for 1 may be selected to be centered around 20 kHz (e.g., 19.5 kHz and 20.5 kHz).

The sensor can include any suitable sensor operative to detect a physiological signal that a user desires to monitor. Nonlimiting examples of such physiological signals include, but are not limited to, respiration, heart beat, heart rate, pulse oximetry, photoplethysmogram (PPG), temperature, etc. A respiration detector can be used. Heart beat and heart rate can be detected as well. For example, the oxygenation of a person's hemoglobin can be monitored indirectly in a noninvasive manner using a pulse oximetry sensor, rather than measuring directly from a blood sample. The sensor is placed on a thin part of the person's body, such as a fingertip or earlobe, and a light containing both red and infrared wavelengths is passed from one side to the other. The change in absorbance of each of the two wavelengths is measured and the difference used to estimate oxygen saturation of a person's blood and changes in blood volume in the skin. A photoplethysmogram (PPG) can then be obtained using the pulse oximeter sensor or with an optical sensor using a single light source. The PPG can be used to measure blood flow and heart rate. A digital representation of this data may then be used and passed on as described herein.

A converter assembly may then convert the digital (electrical) endcoding of the biological parameter to an ultrasound signal that can be transmitted. In the embodiment shown in FIG. 4, the converter assembly includes an ultrasound transducer 407 for outputting ultrasonic signals. Nonlimiting examples of suitable ultrasonic transmitters (including transducers) include, but are not limited to, miniature speakers, piezoelectric buzzers, and the like.

Within the telecommunications device 425, the ultrasonic signals can be received by, for example, a microphone 429 in a device such as a smartphone, personal digital assistant (PDA), tablet personal computer, pocket personal computer, notebook computer, desktop computer, server computer, and the like.

The volume of the signal may be kept low to preserve power, although higher volumes are also possible because the sound is essentially inaudible. For example, the volume of the signal can be further increased at the ultrasonic frequencies, without concern for “listeners” present, because they cannot hear it.

As mentioned above, the telecommunications device may include client logic (e.g., software) for receiving and processing the ultrasound signals. For example, software on the smartphone can decode the ultrasound signal. Processing of the data may provide additional information related to the user including the type of the information (e.g., the nature of the biological parameter. For example: the signal may be encoded so that it contains (after a start identifier): 10 pulses indicating that it is a thermometer reading (e.g., 4 digits coming with last being after the decimal place); 12 pulses indicating it is a blood pressure reading (e.g., 3 digit systolic pressure, 3 digit diastolic pressure and 3 digit pulse rate); 14 pulses indicating that it is pulse oximeter data (e.g., 3 digit 02 sat and 3 digit pulse rate); 16 pulses indicating that it is glucometer data (e.g., 3 digit blood glucose level), etc. There may be a “separator” between the digits and an EOM (end of message) indicator. In practice, the signal may be sent several times so that a comparison may be performed between the received data for validation.

In one variation, the signal may be encoded so that (assuming 8 bit bytes, plus a start and stop bit): some number of AAs, or 55s to allow sync, a byte that denotes a version number, a one byte length of the remainder of the packet, a one byte packet identifier (0x01 for BP, 0x02 for pulse ox, 0x03 for glucose, etc.), data, and an 8-bit CRC.

As mentioned, the signal can have a time and/or date stamp. In some variations the devices or systems may be configured to take multiple measurements and send them to a telecommunications device as a batch or burst. For example, measurements might be made at times t₁, t_{2,} etc., and stored on the device (e.g., thermometer, glucometer, etc.) and transmitted to the telecommunications device (e.g., smartphone, tablet, etc.) ultrasonically at a later time (tₙ). The data may be processed by the telecommunications device and/or uploaded to an external server, etc. (e.g., the cloud).

The baud rate of the transmitted ultrasonic data may be selected to allow rapid transmission. For example, if a baud rate of about 300 baud is used, transmission may take less than a second, even for batched signals. In some variations, the baud rate is around 400.

As mentioned, raw signals from the sensors and derived information can be displayed and stored locally on the smartphone, as well as being transmitted to a web server over an internet connection. Software on the web server may provide a web browser interface for real-time or retrospective display of the signals and information received from the smartphone, and also includes further analysis and reporting.

Ultrasound signaling as used herein refers generally to the transmission of information, such as the magnitude of a biological parameter along with the origin of the biological parameter measurement, using ultrasonic signals. As mentioned, these ultrasonic signals may be encoded to allow transmission and processing. The encoded signal may then be transduced into the ultrasonic range by any appropriate method. For example, one or more frequencies may be used corresponding to various signal values, e.g. DTMF or DTMF frequency-shifted into ultrasonic frequencies. Another example of transducing the signal is to use amplitude shift keying. Another example is to use frequency shift keying. Another example is to use phase shift keying. In some embodiments, multifrequency signaling such as spread spectrum communications, or a multifrequency carrier signaling, may be used. An example of multifrequency carrier signaling is to designate a predetermined set of frequencies (for example, between 20 KHz and 22 KHz, or between 20 KHz and 24 KHz, or generally between a lower bound between 19 KHz and 20 KHz and an upper bound equal to or slightly below the Nyquist frequency for the sampling rate of an intended receiver) separated by an interval, such as an interval of between 40 Hz and 100 Hz, such as approximately 65 Hz, and for each such frequency, encode a “1” bit as the presence of a carrier signal, such as a sine wave at the frequency, and a “0” bit as the absence of such a signal. A receiver of such a multifrequency signal may then perform Fast Fourier Transforms or related techniques known in the art to identify whether carriers are available at each relevant frequency, and deduce a set of bits, encoding a number, thereby. In some embodiments of multifrequency carrier signaling, for example when a signal is insufficiently unambiguous, multiple samples may be taken over time and averaged, then the average signal may be processed as described above. In some embodiments of multifrequency carrier signaling, a Viterbi decoder may be used to decode the bit patterns, for example if the frequencies are sufficiently close as to cause interference. In general, techniques known to those skilled in the communications arts, especially with respect to modulation and demodulation (e.g. modems), may be employed. Examples of such techniques include the various modem standards designated as V.x (where x is an integer) promulgated by the International Telecommunications Union, Sector T, which are incorporated herein in their entirety by reference for all purposes.

In some embodiments, a server may perform signal analysis to determine the encoded data, rather than (or in addition) to on the telecommunications device. In some embodiments, signals may be stored at the server and provided to personnel for refinement of transmission and/or reception techniques.

As mentioned above, signaling may be performed by a transmitter. A transmitter may include a hardware system that incorporates a signal generator such as processor, such as a microprocessor, microcontroller, or digital signal processor connected to a memory (for example, DRAM or SRAM, which in some embodiments may be integrated with the processor) containing program instructions executable by the processor, and/or data used by the program. A transmitter may also incorporate persistent memory, such as a flash memory, coupled to the processor and/or incorporated into the processor. The signal generator may generate the ultrasonic signal that is transmitted as described above. In some embodiments, a waveform for transmission may be stored in persistent memory. In some embodiments, a transmitter includes a power supply and/or a battery, or uses the power supply used to power other components on the medical sensing device. As mentioned, the transmitter may include a transducer, for example a piezoelectric transducer that converts electrical impulses to ultrasonic vibrations. A transmitter may include an amplifier coupled (directly or indirectly, for example via an audio Digital-to-Analog Converter (DAC), which in some embodiments may be integrated with the processor) to the processor, which provides electrical impulses through its output to the transducer. In some embodiments, transmitter may include a real-time clock and/or a receiver for receiving broadcast time signals. In some embodiments, transmitter may include an encryptor, which for example may be program instructions executing on processor, or may be separate integrated circuitry. In some embodiments, transmitter may include an error correcting code generator and/or an error detecting code generator, which for example may be software instructions executing on processor, or may be separate integrated circuitry. The techniques described herein regarding transmission and reception of sonic signaling may be performed at a transmitter as described herein in a manner that will be readily understood by those skilled in the art.

In some variations, the transmission from the medical sensing device to the telecommunications device is one-way. This configuration is desirable because it may allow a number of previously unrealized advantages, including the simplicity of the design, lower expense, lower power consumption, and the like. These advantages are particularly true when compared to systems in which the medical sensing device includes an additional receiver (including a microphone for receiving sonic signals, or an antenna). However, in some configurations the medical sensing device may be adapted to receive a simple indicator signal from the telecommunications device without the addition of a receiver such as an antenna or microphone. For example, in some variations a return acknowledgement (ACK) could be implemented using the ultrasonic transducer (e.g., piezo speaker) as a 20 khz sensor. For example, the telecommunications device (e.g., phone) could produce a short 20 khz burst after receiving, decoding, and verifying the CRC to signal to the sensor that it received it correctly, indicating that re-transmission is not necessary. In other variations a signal from the telecommunications device may indicate that it is ready to receive transmission from the biometric device. Pairs or multiples of timed signals/acknowledgements may also be used.

In one example, the devices or systems are configured so that the data that is ultrasonically transmitted includes forward error correction (FEC), allowing the receiver to correct N number of bit errors. This may be particularly useful if the system is configured so that the biometric device (the medical sensing device) is transmit-one (e.g., one-way). FEC may help ensure that the data is received correctly.

In some embodiments, data sent by ultrasonic signaling may be processed to include an error correcting code, such as a BCH code, a Constant-weight code, a Convolutional code, a Group code, a Golay code such as a Binary Golay code, a Goppa code, a Hadamard code, a Hagelbarger code, a Hamming code, a Latin Square based code, a Lexicographic code, a sparse graph code such as a Low-Density Parity-Check code, an LT or “Fountain” code, an Online code, a Raptor code, a Reed-Solomon code, a Reed-Muller code, a Repeat-accumulate code, a Repetition code such as Triple modular redundancy code, a Tornado code, a Turbo code, or other error correcting codes known to those skilled in the art. In various embodiments, such codes may be applied in a single dimension or in multiple dimensions, may be combined, and may be combined with error detecting codes such as parity and cyclic redundancy checks. Error correcting codes may be decoded and applied to correct transmission and/or reception errors at a receiver, or at a server receiving communications from a receiver, according to their respective techniques.

### EXAMPLE 1

### Digital Thermometer

In one example, a digital thermometer may be configured to include a digital ultrasonic modem. In this example, a digital thermometer based on a Texas Instrument MSP430 digital thermometer has been adapted to include firmware so that it may ultrasonically transmit the temperature reading (digital data) to a mobile telecommunications device (e.g., iPhone). Although this example is specific to the APE 4110 microprocessor (one variation of the MSP 430 microprocessor from Texas Instruments) other microprocessors may be used and similarly adapted with firmware, software and/or hardware to function.

In general, the device may take data (e.g., thermometer temperature readings) and encode them for ultrasonic transmission. The encoded signal may include error checking (e.g., CRC encoding, Hamming codes, etc.) and may be encrypted. For example, the data may be data encrypted using, for example Advanced Encryption Standard (AES). U.S. Pat. Nos. 5,481,255 and 5,452,356 both describe data encryption methods and techniques that may be used with the data described herein.

For example, data received from the thermometer may be encoded and/or encrypted into one or more data packets for transmission. The microprocessor may encode the data and may then transmit the packets by driving the piezo speaker. As mentioned above Frequency Shift Keying (FSK) may be used, in which two separate ultrasonic frequencies (e.g., 18817 Hz and 19672 Hz) are used to transmit Boolean 0 and 1, respectively. The control logic (data ultrasound modem logic) may both configure, encode and encrypt the data and may also control driving the transmission of the prepared packets of encoded/encrypted data by the speaker (e.g., piezoelectric transducer). The control logic may also control the timing of the delivery, so that there is adequate spacing between each data bit. In addition, the control logic may also repeat the transmission and time the start of the transmission.

For example, in one variation the thermometer typically measures temperature, and once the temperature has settled to a value, the thermometer emits an audible beep to alert the user that the value can be read. This thermometer (in the initially unmodified configuration) includes a microcontroller (e.g., the AFE 4110) and a piezoelectric speaker; the microcontroller drives the speaker to emit the beep. By modifying/configuring the microcontroller as described herein to include the control logic for the digital ultrasound modem, the thermometer may be adapted to “wirelessly” (via ultrasound) transmit the thermometer data to a device configured to receive and decode/decrypt the signal such as a smartphone running digital ultrasound modem receiver logic.

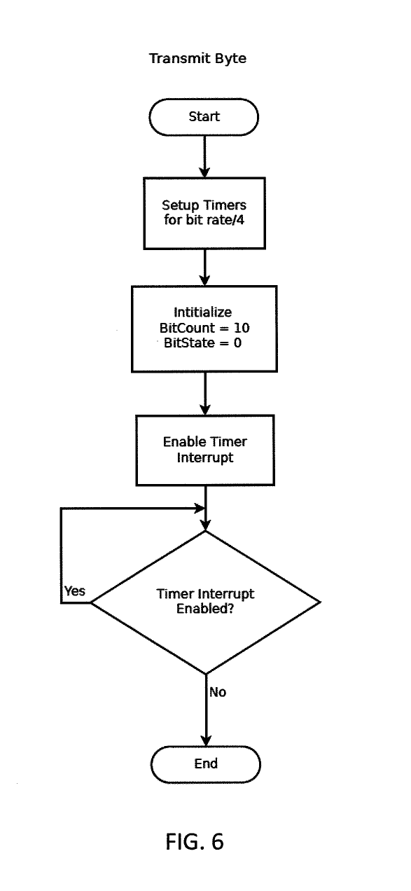

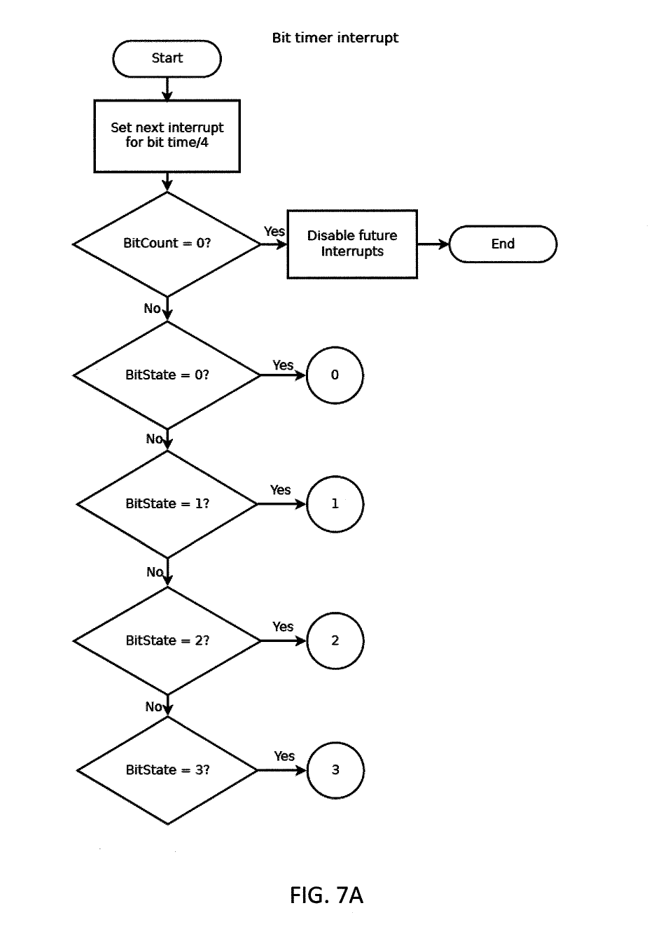

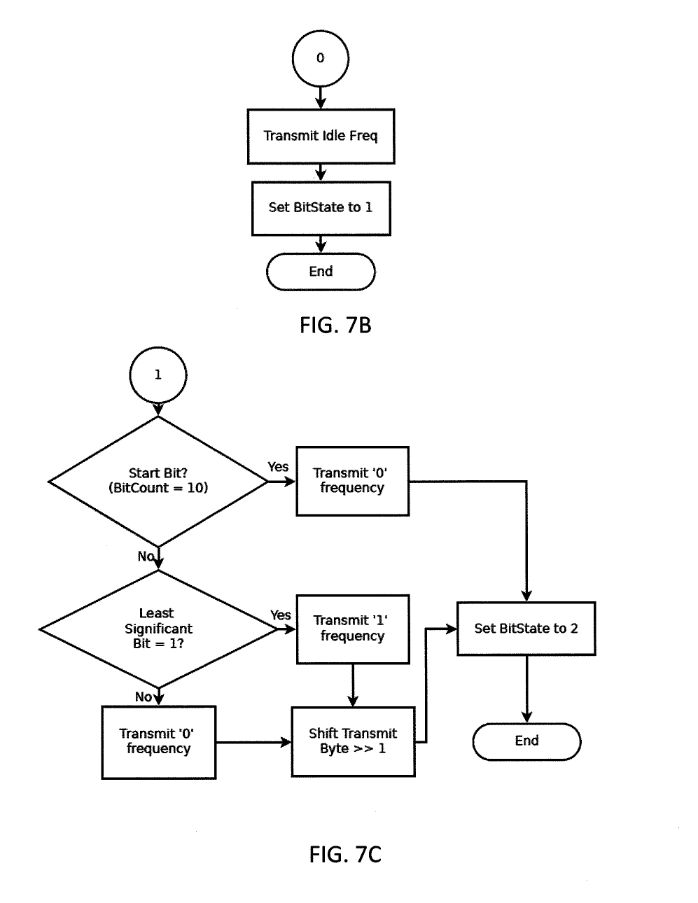

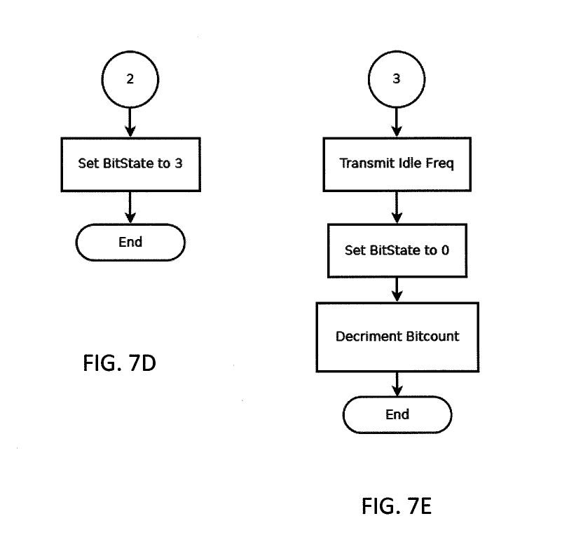

In this example, the microprocessor may include the following (exemplary) code to enable the functionality described above. FIGS. 6 and 7A-7E show flowcharts describing methods for transmitting data. Exemplary control logic follows:

```
  // Transmit byte and add it to the CRC calculation
  unsigned short TransmitWithCRC(unsigned char ByteToSend,
unsigned short CRC)
  {
      Transmit(ByteToSend);
      return CalcCRC(ByteToSend, CRC);
  }
  // Transmits given temperature using FSK
  int TransmitTemp(int TempInC)
  {
      unsigned short CRC = 0xFFFF; // Initial CRC Value
    // The following 5 bytes are not included in the CRC
    // calc, and are not transmitted Hamming encoded
      TransmitEncoded(0x55);
      TransmitEncoded(0x55);
      TransmitEncoded(0x55);
      TransmitEncoded(0x00);
      TransmitEncoded(0xFF);
    // Start of payload
```

| | |
|---|---|
| CRC = TransmitWithCRC(0x02, CRC); | // Version |

number of the rest of this packet

| | |
|---|---|
| CRC = TransmitWithCRC(0x03, CRC); | // Length |

of the rest of the packet

| | |
|---|---|
| CRC = TransmitWithCRC(0x00, CRC); | // Packet |

```
identifier for temperature
      CRC = TransmitWithCRC(((TempInC & 0xFF00) >> 8), CRC);
      CRC = TransmitWithCRC((TempInC & 0x00FF), CRC);
    // End of payload
```

| | |
|---|---|
| Transmit((CRC & 0xFF00) >> 8); | // |

Transmit upper byte of CRC

| | |
|---|---|
| Transmit(CRC & 0x00FF); | // |

```
Transmit lower byte of CRC
      return 0;
  }
  // Compile time calculate timer values
  #define CLK_RATE 4328000
  #define CC_VAL(S) CLK_RATE/S
```

| | |
|---|---|
| #define FSK_0_CC0 CC_VAL(18817) | // 18817Hz for |

FSK 0
#define FSK_0_CC1 FSK_0_CC0/2

| | |
|---|---|
| #define FSK_1_CC0 CC_VAL(19672) | // 19672Hz for |

FSK 1
#define FSK_1_CC1 FSK_1_CC0/2

| | |
|---|---|
| #define FSK_IDLE_CC0 CC_VAL(21640) | // 21640Hz for |

```
Idle, and guard periods
  #define FSK_IDLE_CC1 FSK_IDLE_CC0/2
  // Routines to set timers to produce the three frequencies
previously calculated
  //
  #define Transmit0( ) TA0CCR0 = FSK_0_CC0; TA0CCR1 =
FSK_0_CC1
  #define Transmit1( ) TA0CCR0 = FSK_1_CC0; TA0CCR1 =
FSK_1_CC1
  #define TransmitIdle( ) TA0CCR0 = FSK_IDLE_CC0; TA0CCR1 =
FSK_IDLE_CC1
  // Transmit one byte fsk with Hamming encoding
  int Transmit(unsigned char BytetoTransmit)
  {
    int retval = −1;
    // Transmit lower nibble+parity first
    retval =
TransmitEncoded(HammingTableEncode(BytetoTransmit & 0x0F));
    if ( retval < 0 )
    {
        return retval;
    }
    // Then transmit upper nibble+parity
    retval =
TransmitEncoded(HammingTableEncode((BytetoTransmit & 0xF0) >> 4));
    return retval;
  }
  // Transmit 8 bits that is already Hamming encoded
  int TransmitEncoded(unsigned char BytetoTransmit)
  {
    // Start transmitting the start bit
    // setting timer0 clock to SMCLK, up count, no
interrupts
    TA0CTL = MC_UP | TASSEL_SMCLK;
    BitCount = 10;
    Bitstate = 0;
    TA1R = 0;
```

| | |
|---|---|
| TA1CCR0 = TA1R + (BIT_TIME/4); | // Bit time is |

```
divided by four to allow guard periods
    TransmitIdle( );
    // setting timer1 clock to SMCLK, up count, interrupts
    TA1CTL = MC__CONTINOUS | TASSEL_SMCLK;
    // Add Stop bit;
    TxByte = (unsigned int)BytetoTransmit | 0x100;
    // Enable timer1 interrupt
    TA1CCTL0 = CCIE;
    // Wait for ISR to transmit
    while ( TA1CCTL0 & CCIE );
    // Return number of bits transmitted
    return 8;
  }
  // Timer 1 A0 intterupt routine
  #pragma vector = TIMER1_A0_VECTOR
  __interrupt void timerA0(void)
  {
    TA1CCR0 += BIT_TIME/4;
    // Test if we are done with the byte
    if ( BitCount == 0 )
    {
        // If we are done, disable this interrupt to signal
        // to the transmit routine that we are done
        TA1CCTL0 &= ~CCIE;
        TA1CTL &= ~MC_CONTINOUS;
        TA0CTL = TASSEL_SMCLK;
    }
    // Inter-bit state machine
    // First 1/4 of bit period transmit idle
    // Second and third quarters transmit the bit
    // Last quart transmit idle
    switch ( Bitstate )
    {
    case 0:
        TransmitIdle( );
        Bitstate = 1;
        break;
    case 1:
        if ( BitCount == 10 ) // If start bit
        {
          // Start bit is a 0
          Transmit0( );
        }
        else
        {
          if ( TxByte & 0x0001 )
          {
            Transmit1( );
          }
          else
          {
            Transmit0( );
          }
          TxByte = TxByte >> 1;
        }
        Bitstate = 2;
        break;
    case 2:
        Bitstate = 3;
        break;
    case 3:
        TransmitIdle( );
        BitCount--;
        Bitstate = 0;
        break;
    }
  }
  // Hamming encoding routines
  // number of uncoded data bits and data values
```

| | |
|---|---|
| #define DATA_BITS | 4 |
| #define DATA_VALUES | (1 << DATA_BITS) |

```
  // table of Hamming codes hammingCodes[x] is the x encoded
  const unsigned char hammingCodes[DATA_VALUES] =
  {
```

| | |
|---|---|
| 0x00, | /* 0 */ |
| 0x71, | /* 1 */ |
| 0x62, | /* 2 */ |
| 0x13, | /* 3 */ |
| 0x54, | /* 4 */ |
| 0x25, | /* 5 */ |
| 0x36, | /* 6 */ |
| 0x47, | /* 7 */ |
| 0x38, | /* 8 */ |
| 0x49, | /* 9 */ |
| 0x5A, | /* A */ |
| 0x2B, | /* B */ |
| 0x6C, | /* C */ |
| 0x1D, | /* D */ |
| 0x0E, | /* E */ |
| 0x7F | /* F */ |

```
  };
  // HammingTableEncode: This function uses a lookup table to
determine the
```

| | |
|---|---|
| // | Hamming code for a DATA_BITS long |

```
value.
  unsigned char HammingTableEncode(unsigned char data)
  {
    return hammingCodes[data];
  }
```

### Ultrasound Digital Modem Receiver

As mentioned above, a receiver (a digital ultrasound modem receiver) may be used to receive the transmitted ultrasound signal. The receiver may be a dedicate device include a microphone competent to receive ultrasound signals and a processor capable of analyzing the signal (e.g., microprocessor) or it may be a device having microprocessor and microphone that is adapted to receive the ultrasound signal when executing control logic (e.g., digital ultrasound modem receiver logic).

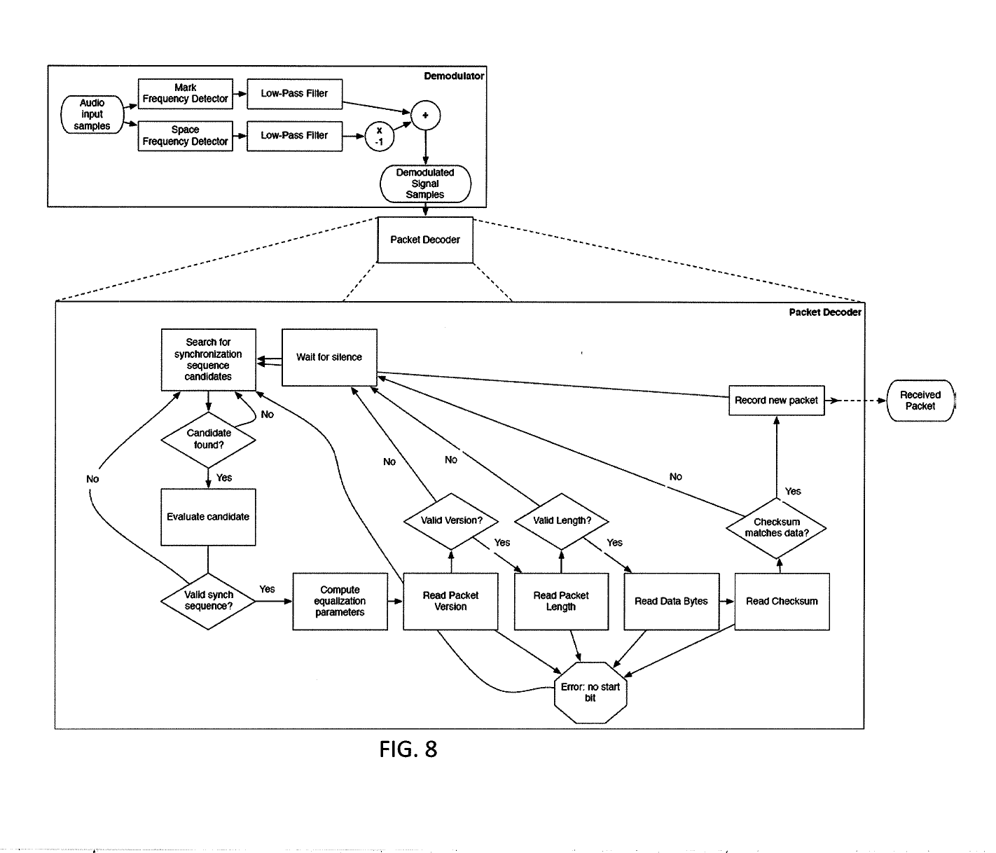

For example, FIG. 8 illustrates one variation of a flow diagram illustrating a method for receiving, demodulating and detecting the digital ultrasound signal. In this example, the application (the receiving control logic) receives binary-FSK encoded data via a microphone input. For example, the input may be from the microphone on a smartphone. As discussed above, Binary FSK encoding uses two frequencies, a “mark” frequency Fₘ to represent a binary 1, and a “space” frequency Fₛ to represent a binary 0. In this implementation, no carrier is used.

The application consists of two largely independent components: the demodulator, which extracts the mark and space frequency components from the raw audio data, and the packet decoder, which monitors the demodulated signal for packet transmissions and decodes them. These are illustrated in FIG. 8. The demodulator receives audio samples from the microphone hardware at a sample rate S, such that S>2*max(Fₘ,Fₛ). The audio samples are processed by two frequency detectors that calculate the intensity of the mark and space frequency components (respectively) of the received signal. A Goertzel algorithm is used for frequency detection in this implementation. In order to achieve sufficient frequency resolution between the mark and space frequencies, we apply the Goertzel algorithm to a sliding window of G samples, where G=S/abs(Fₘ−Fₛ).

The output of the Goertzel algorithm for the mark and space frequencies is passed to independent low-pass filters, with a passband equal to the baud rate. The filtered output of the space frequency signal is then subtracted from the filtered output of the mark frequency signal. This produces a waveform that is approximately 0 when there is no transmission occurring, rises to a positive value when the “mark” frequency is active, and falls to a negative value when the “space” frequency is active.

This demodulated waveform is then passed to the packet decoder. For each raw audio sample received from the microphone hardware, the demodulator produces a single demodulated sample of the demodulated waveform. The packet decoder receives demodulated samples from the demodulator. The decoder maintains a buffer of the last N samples received, where N is equal to the length of the synchronization sequence. With each new sample, the decoder evaluates the past N samples in the buffer to determine if they contain the synchronization sequence. A two-stage test is used—first a computationally simple evaluation that eliminates most false positives due to random noise, and then a more computationally expensive evaluation that eliminates the rest.

Once a valid synchronization sequence is received, the decoder stores properties of the received signal (e.g. maximum mark/space amplitudes, etc.). These equalization parameters are used to calibrate the decoder thresholds used to read the remainder of the packet. The decoder in this example then reads each encoded byte in turn. It uses the stored equalization parameters to determine a minimum amplitude threshold for the start bit of each byte. Once a valid start bit is received for a given byte, subsequent bits are evaluated based on the sign of the demodulated waveform, with no minimum threshold for decoding.

If no valid start bit is received, the decoder aborts reading the packet and waits for silence, or until a fixed amount of time has passed, before resuming listening for new packets. Each logical byte in the packet is actually transmitted as two encoded bytes—the first containing the Hamming-encoded low nibble of the logical byte, and the second the Hamming-encoded high nibble.

The first logical byte read is the packet version, which is checked against supported version numbers. Next the packet length is read, specifying the number of data bytes to follow. If the packet length exceeds the maximum length for the specified packet version, the packet is rejected. Subsequently, each logical data byte is read.

After the data bytes are read, two logical checksum bytes are read, and the checksum value received is compared to the value computed for the data bytes received. If these two checksum values match, the packet is considered valid, and is made available to the remainder of the application. If they do not match, the packet is rejected. The two logical checksum bytes represent the end of the packet. After receiving the packet, the decoder resumes listening for new packets.

Once data is received (and in some variations decrypted), it may be processed further and/or stored, and/or displayed, and/or transmitted on using any of the communications capabilities of the telecommunications device. For example, the data may displayed on the smartphone and also uploaded into a medical database for storage and/or later review.

Although the systems described herein are configured to transmit digital information, the techniques, device and systems described herein may be configured to transmit analog signals as well. In general, the techniques described include the use of a timer (e.g., in the microcontroller) transmitting to a piezo to generate the ultrasound signal. Alternatively, in some variations the system uses a D/A converter to drive a speaker for non-digital output. Further, in some variations the system the output is not a piezoelectric element but is a more traditional speaker (albeit in the ultrasound range). Additional digital to analog (D/A) conversions may take place during transmission.

From the above descriptions, it is clear that the presently disclosed and claimed inventive concept(s) are well-adapted to carry out the objects and to attain the advantages mentioned herein, as well as those inherent in the presently disclosed and claimed inventive concept(s). While the presented embodiments have been described for purposes of this disclosure, it will be understood that numerous changes may be made which will readily suggest themselves to those skilled in the art and which are accomplished within the spirit of the presently disclosed and claimed inventive concept(s).

What may be claimed is:

1. A medical sensing device for detecting a biological parameter, determining a digital representation of the biological parameter, and ultrasonically transmitting the digital representation of the biological parameters as an inaudible sound transmission, the device comprising:
a sensor for detecting a biological parameter from a subject;
a processor configured to receive the biological parameter, determine a representative value from the biological parameter, and digitally encode the representative value as a digital ultrasound signal, wherein the digital ultrasound signal is encoded using a first frequency corresponding to digital zero and a second frequency corresponding to digital 1, wherein the first and second frequencies are each greater than 17 kHz, further wherein the digital ultrasound signal includes a header portion and a data portion; and
an ultrasonic transducer comprising an ultrasound emitter for transmitting the digital ultrasound signal, wherein the processor is configured to drive the ultrasonic transducer to emit the digital ultrasound signal from the ultrasound emitter.

2. The device of claim 1, wherein the sensor is configured to detect one or more of:
temperature, glucose, pulse oxygenation, or blood pressure.

3. The device of claim 1, wherein there processor is a microprocessor.

4. The device of claim 1, wherein the first frequency is approximately 18.5 kHz and the second frequency is approximately 19.5 kHz.

5. The device of claim 1, wherein the processor is configured to digitally encode the digital ultrasound signal at 10 cycles per bit.

6. The device of claim 1, wherein the processor is configured to digitally encode the digital ultrasound signal at 200 bytes/second.

7. The device of claim 1, wherein the processor is further configured to send a calibration tone at a frequency that is separate from the first and second frequencies.

8. The device of claim 1, wherein the digital ultrasound signal includes an error correction code portion.

9. The device of claim 1, wherein the ultrasound emitter comprises a speaker.

10. The device of claim 1, wherein the ultrasound emitter comprises a piezoelectric element.

11. A system for detecting a biological parameter, determining a digital representation of the biological parameter, and ultrasonically transmitting the digital representation of the biological parameters as an inaudible sound transmission, the system comprising:
a medical sensing device having: a sensor for detecting a biological parameter, a processor configured to receive the biological parameter, determine a representative value from the biological parameter, and digitally encode the representative value as a digital ultrasound signal using a first frequency corresponding to digital zero and a second frequency corresponding to digital 1, wherein the first and second frequencies are each greater than 17 kHz, and an ultrasonic transducer for transmitting a digital ultrasound signal; and
client control logic configured to be executed by a telecommunications device and to cause the telecommunications device to receive the digital ultrasound signal and extract the representative value of the biological parameter from the digital ultrasound signal.

12. The system of claim 11, wherein the wherein the sensor is configured to detect one or more of: temperature, glucose, pulse oxygenation, or blood pressure.

13. The system of claim 11, wherein there processor is a microprocessor.

14. The system of claim 11, wherein the first frequency is approximately 18.5 kHz and the second frequency is approximately 19.5 kHz.

15. The system of claim 11, wherein the processor is configured to digitally encode the digital ultrasound signal at 10 cycles per bit.

16. The system of claim 11, wherein the processor is configured to digitally encode the digital ultrasound signal at 200 bytes/second.

17. The system of claim 11, wherein the processor is further configured to send a calibration tone at a frequency that is separate from the first and second frequencies.

18. The system of claim 11, wherein the digital ultrasound signal includes a header portion, a data portion and an error correction code portion.

19. The system of claim 11, wherein the client control logic comprises non-transitory computer-readable storage medium storing a set of instruction capable of being executed by a smartphone.

20. The system of claim 11, wherein the ultrasound emitter comprises a piezoelectric element.

21. A digital thermometer to ultrasonically transmit digital temperature information to a telecommunications device for further processing and transmission, the digital thermometer comprising:
a temperature sensor for sensing subject's temperature;
a processor in communication with the temperature sensor and configured to generate a digital ultrasound signal of the subject's temperature, wherein the digital ultrasound signal is encoded using a first frequency corresponding to digital zero and a second frequency corresponding to digital 1, wherein the first and second frequencies are each greater than 17 kHz; and
an ultrasonic transducer comprising an ultrasound emitter, wherein the processor is configured to drive the ultrasonic transducer to emit the digital ultrasound signal from the ultrasound emitter.

22. The device of claim 21, wherein there processor is a microprocessor.

23. The device of claim 21, wherein the first frequency is approximately 18.5 kHz and the second frequency is approximately 19.5 kHz.

24. The device of claim 21, wherein the processor is configured to digitally encode the digital ultrasound signal at 10 cycles per bit.

25. The device of claim 21, wherein the processor is configured to digitally encode the digital ultrasound signal at 200 bytes/second.

26. The device of claim 21, wherein the processor is further configured to send a calibration tone at a frequency that is separate from the first and second frequencies.

27. The device of claim 21, wherein the digital ultrasound signal includes a header portion, a data portion and an error correction code portion.

28. The device of claim 21, wherein the ultrasound emitter comprises a speaker.

29. The device of claim 21, wherein the ultrasound emitter comprises a piezoelectric element.

30. A method of locally transmitting a representative value of a biological parameter using ultrasound, the method comprising:
sensing a biological parameter from a subject;
determining a representative value from the biological parameter;
digitally encoding the representative value as a digital ultrasound signal, wherein the digital ultrasound signal is encoded using a first frequency corresponding to digital zero and a second frequency corresponding to digital 1, wherein the first and second frequencies are inaudible ultrasound frequencies; and
driving an ultrasonic transducer near the patient to emit the digital ultrasound signal as an inaudible sound signal.

31. The method of claim 30, wherein sensing a biological parameter comprises sensing one or more of: temperature, glucose, pulse oxygenation, or blood pressure.

32. The method of claim 30, wherein determine a representative value comprises determining one or more of an average, a mean, a median, a maximum, a minimum, or a rate of change.

33. The method of claim 30, wherein digitally encoding the representative value comprises encoding the digital ultrasound signal to include a header portion and a data portion.

34. The method of claim 30, wherein digitally encoding the representative value comprises encoding the digital ultrasound signal to include a header portion, a data portion, and an error correction code portion.

35. The method of claim 30, wherein the first frequency and the second frequency are each greater than 17 kHz.

36. The method of claim 30, wherein digitally encoding the representative value comprises digitally encoding the digital ultrasound signal at 10 cycles per bit.

37. The method of claim 30, wherein digitally encoding the representative value comprises digitally encoding the digital ultrasound signal at 200 bytes/second.

38. The method of claim 30, further comprising emitting a calibration tone at a frequency that is separate from the first and second frequencies.

39. The method of claim 30, further comprising repeatedly driving the ultrasonic transducer to emit the digital ultrasound signal until a receipt confirmation is received.

40. The method of claim 30, further comprising repeatedly driving the ultrasonic transducer to emit the digital ultrasound signal for a predetermined period of time or number of repeats.

41. An integrated microprocessor configured as an local ultrasonic data transmission device, the microprocessor comprising
a non-transitory computer-readable storage medium storing a set of instruction for: receiving a value, digitally encoding the value as a digital ultrasound signal, wherein the digital ultrasound signal is encoded using a first frequency corresponding to digital zero and a second frequency corresponding to digital 1, wherein the first and second frequencies are inaudible ultrasound frequencies, adding a header portion to the digital ultrasound signal; and
an ultrasonic transducer comprising an ultrasound emitter for transmitting the digital ultrasound signal.
